(19) United States
(12) Patent Application Publication
(10) Pub. No.: US 2020/0281624 A1
(43) Pub. Date: Sep. 10, 2020

(54) DEVICES AND METHODS FOR SECURING SURGICAL GUIDE WIRES

(71) Applicant: SECURE SURGICAL INC, Asheville, NC (US)

(72) Inventor: David J. Hetzel, Asheville, NC (US)

(21) Appl. No.: 16/881,121

(22) Filed: May 22, 2020

Related U.S. Application Data
(63) Continuation-in-part of application No. 15/267,935, filed on Sep. 16, 2016, now Pat. No. 10,688,286.
(60) Provisional application No. 62/219,848, filed on Sep. 17, 2015.

(51) Int. Cl. A61B 17/34; G16H 40/67; G16H 10/65

(52) U.S. Cl. CPC A61B 17/3403; G16H 40/67; A61B 2017/00349; A61B 2017/3407; A61B 2017/3413; G16H 10/65

(57) ABSTRACT

Provided herein are devices and methods for securing surgical guide wires. In particular, provided herein are devices for securing radiologically located guide wires prior to surgery (e.g., lumpectomy) and associated medical device communication system and methods.

This application is a continuation in part of U.S. patent application Ser. No. 15/267,935, filed Sep. 16, 2016, which claims priority to provisional application 62/219,848, filed Sep. 17, 2015, which is herein incorporated by reference in its entirety.

### FIELD OF THE INVENTION

Provided herein are devices and methods for securing surgical guide wires. In particular, provided herein are devices for securing radiologically located guide wires prior to surgery (e.g., lumpectomy) and associated medical device communication system and methods.

### BACKGROUND OF THE INVENTION

Needle localizations are commonly performed by radiologists before excisional biopsy of non-palpable breast lesions, using one of a number of commercially available needle and wire systems such as the Kopans wire. An imaging device such as an ultrasound probe, mammogram, CT, or MM is used to place the wire in or around the abnormal area. The surgeon then uses this wire as a guide to find and remove the tumor during surgery. The wire will also be removed during surgery.

After wire localization, patients then have to travel, often in a car to the hospital or outpatient surgery center. During transit, it is important that wires do not move or become dislodged. If the wires are not properly localized, a delay in surgery and costly replacement of the wires is often required.

The long, protruding wire is difficult to manage. Currently, wires are typically covered with a bandage and/or medical tape, which does not provide adequate protection against wires dislodging. Improved methods of securing guide wires are needed.

### SUMMARY OF THE INVENTION

Provided herein are devices and methods for securing surgical guide wires. In particular, provided herein are devices for securing radiologically located guide wires prior to surgery (e.g., lumpectomy) and associated medical device communication system and methods.

For example, in some embodiments, provided herein is a guide wire securing device, comprising: one or more of a wire conformation, retention, and management component (e.g., one or more of wire hooks and/or a grooved track), a locking component (e.g., comprising one or more of a clocking tab insertion component, a locking tab, a connecting arm incorporating a hinge (e.g., living hinge), a wire insertion groove, a locking clip, and a wire hole, or an attachment component). In some embodiments, the wire insertion groove is Y-shaped. In some embodiments, the locking tab comprises a rubber washer and closing tab. In some embodiments, the attachment component comprises adhesive attached to the back edge of the device. In some embodiments, the adhesive is foam adhesive. In some embodiments, the device is fabricated from a polymeric material.

Further embodiments provide systems and kits comprising the devices described herein and a plurality of guide wires (e.g., surgical guide wires).

Additional embodiments provide a method of securing a guide wire, comprising: a) contacting a guide wire inserted into a body part of a patient with a guide wire securing device, comprising: a wire conformation, retention, and management component, a locking component, and an attachment component; b) inserting the guide wire through the wire retention component of the device; and c) securing the wire using the locking component. In some embodiments, the guide wires are surgical guide wires (e.g., breast cancer guide wires that identify a breast lump or tumor). In some embodiments, the device is securing to the patient using the adhesive component. In some embodiments, the patient is pre-operative. In some embodiments, the device is removed from the patient prior to the patient undergoing surgery (e.g., lumpectomy). In some embodiments, the guide wires protrude from the patient and are long (e.g., approximately 7-22 cm). In some embodiments, the device facilities rapid and easy wrapping and management of the guide wires.

Additional embodiments provide guide wire securing device (e.g., as described herein), comprising: a computer readable medium comprising a) a wireless communications protocol and one or more of a) information about the device; b) images of the device; and c) a device specific identification number that pairs with a remote server.

The present disclosure is not limited to particular information. Examples include, but not limited to, device ID, device age, device brand, device class and use parameters, patient information (e.g., one or more of diagnosis information and treatment information), or device model. In some embodiments, the images comprise images of the device in place in a subject.

In some embodiments, the computer readable medium comprises a communication component selected from, for example, a radiofrequency identification chip, a near field communication chip, a blue tooth communication chip, or a WiFi communication chip. In some embodiments, the information is stored on the computer readable medium or on a remote server. In some embodiments, the computer readable medium is integrated into an external or internal portion of the device. In some embodiments, the information is stored in a read-only format.

Further embodiments provide a system, comprising: a) a device as described herein; and b) a personal electronic device configured to access information stored on the device.

The present disclosure is not limited to particular personal devices. Examples include, but are not limited to, a smart phone, smart watch, tablet, or laptop computer.

Additional embodiments include a method of obtaining information, comprising: a) contacting a device as described herein with a personal electronic device; and b) accessing information stored on the medical device with the personal electronic device using the wireless communication protocol. In some embodiments, the method further comprises providing a patient specific passcode to the personal electronic device. In some embodiments, the passcode is required for access to the information.

Yet other embodiments provide a method of storing and retrieving medical device information, comprising: a) storing information on a device as described herein comprising a computer readable medium comprising a wireless communications protocol, wherein the information is selected from, for example, patient information; information about the device; medical images of the device and associated guide wires in place with a specific patient or subject; or a device specific identification number that pairs with a remote server; b) providing the device to a subject; and c) contacting the device with a personal electronic device to access information stored on the medical device using the wireless communication protocol.

Still further embodiments provide a method of providing medical care or treatment to a subject, comprising: a) storing information on a device comprising a computer readable medium comprising a wireless communications protocol, wherein the information is selected from, for example, patient information; information about the device; images of the device in vivo in the specific patient; or a device specific identification number that pairs with a remote server; b) providing the device to the subject; c) contacting the device with a personal electronic device to access information stored on the medical device using the wireless communication protocol; d) determining an appropriate approach to surgical treatment or medical care based on the information (e.g., whether or how to proceed with surgery); and e) administering the treatment or medical care to the subject. In some embodiments, the contacting occurs at a different geographical location than the storing.

Additional embodiments are described herein.

### DESCRIPTION OF THE FIGURES

FIG. 1 shows a top view of exemplary devices of embodiments of the present disclosure.

FIG. 2 shows a back view of exemplary devices of embodiments of the present disclosure.

FIG. 3 shows a side view of exemplary devices of embodiments of the present disclosure.

FIG. 4 shows a close up of the locking area of exemplary devices of embodiments of the present disclosure.

FIG. 5 shows a close up of the locking area of exemplary devices of embodiments of the present disclosure.

FIG. 6 shows a close up of the inner track for wrapping and securing wires of exemplary devices of embodiments of the present disclosure.

FIG. 7A and FIG. 7B show an exemplary device of embodiments of the present disclosure with guide wires attached.

FIG. 8 shows an exemplary device of embodiments of the present disclosure with guide wires attached and secured.

FIG. 9A and FIG. 9B show an exemplary device of embodiments of the present disclosure with guide wires attached and secured and the locking tab closed.

FIG. 10 shows an exemplary device of embodiments of the present disclosure with guide wires attached and secured and wrapped around the wire hooks.

FIG. 11 shows an exemplary device of embodiments of the present disclosure with guide wires attached and secured and wrapped around the wire hooks.

FIG. 12 shows a flow chart of an exemplary work-flow for retrieval of information using a system of embodiments of the present disclosure.

FIG. 13 shows a flow chart of an exemplary work-flow for retrieval of information using a system of embodiments of the present disclosure.

FIG. 14A-FIG. 14D show exemplary devices of embodiments of the present disclosure with CRM chips.

### DESCRIPTION OF THE INVENTION

Provided herein are devices and methods for securing surgical guide wires. In particular, provided herein are devices for securing radiologically located guide wires prior to surgery (e.g., lumpectomy) and associated medical device communication system and methods.

Embodiments of the present disclosure provide devices and methods that improve over existing methods of securing guide wires after placement. In some embodiments, the present disclosure provides devices that serve to securely lock the wires in place and prevent movement or dislodging of wires. The devices and methods described herein assist the patient and clinicians in managing the wire in a quicker and easier manner than existing technologies.

For example, in some embodiments, provided herein is a guide wire securing device, comprising: one or more of a wire conformation, retention, and management component (e.g., one or more of wire hooks and/or a grooved track), a locking component (e.g., comprising one or more of a clocking tab insertion component, a locking tab, a hinge, a wire insertion groove, a locking clip, and a wire hole, or an attachment component).

As used herein, the term “wire retention and management component” refers to a component of the described devices that serves to secure wires and management the conformation, location, and security of wires (e.g., guide wires). In some embodiments, the wire retention and management component comprises one or more components including, but not limited to, wire hooks, a grooved wire track, and the like.

As used herein, the term “locking component” refers to a component of the devices described herein that locks wires (e.g., wires secured by the “wire retention and management component”) in place (e.g., during use of the guide wires). In some embodiments, the locking component comprises one or more components including, but not limited to, a clocking tab insertion component, a locking tab, a hinge, a wire insertion groove, a locking clip, a wire hole, and the like.

The term “living hinge” refers to a hinge (e.g., as described herein) that comprises a plurality (e.g., 1, 2, 3, 4, or more) grooves in a connecting arm that allow the arm to easily bend without breaking. In some embodiments, this allows the locking cap to bend about 180 degrees over and into snapped down or locked position.

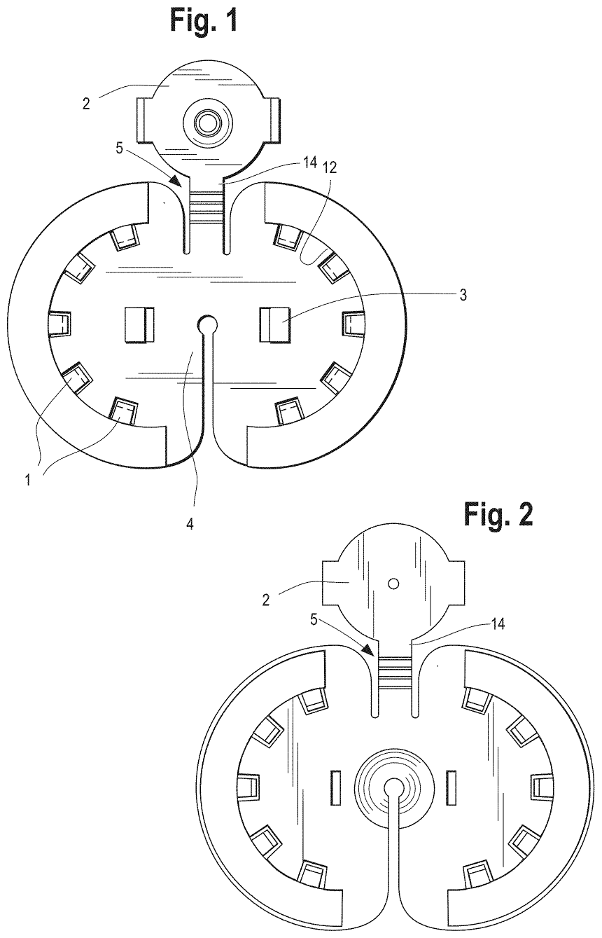

Exemplary devices of embodiments of the present disclosure are shown in FIGS. 1-6. FIG. 1 shows a top view of an exemplary device. Shown are wire hooks 1; locking component comprising locking tab 2, locking clip 3, and locking tab insertion component 4; grooved track 12, and a connecting arm 14 incorporating hinge 5. In some embodiments, the hinge is a living hinge. In some embodiments, the living hinge comprises bendable sections 13.

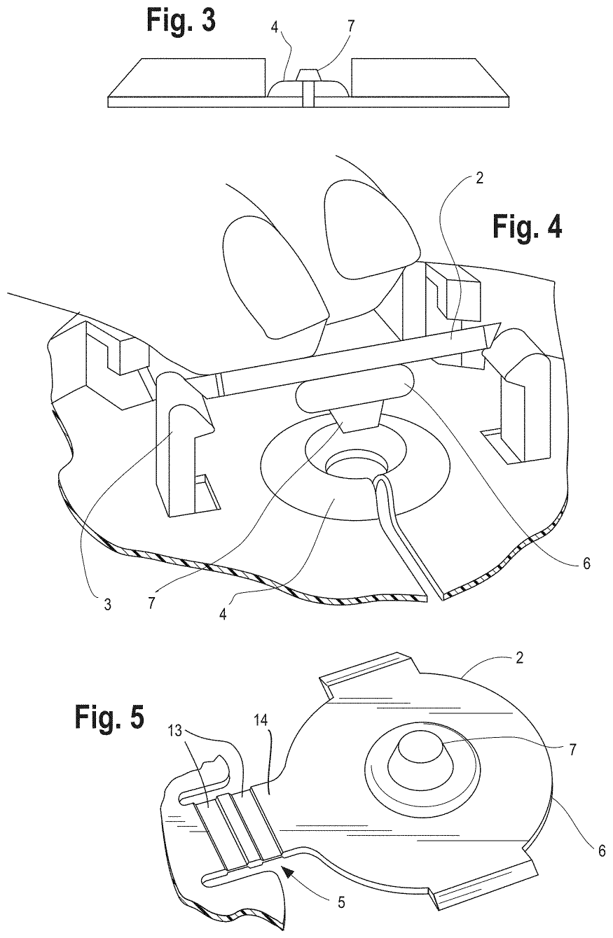

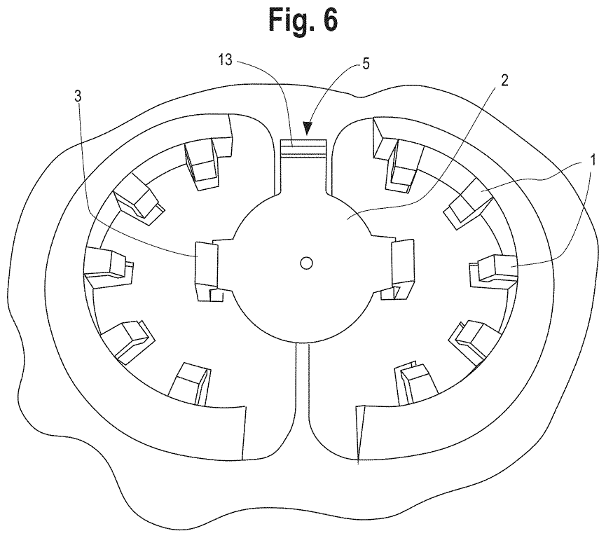

FIG. 2 shows a back view of an exemplary device. Shown are wire hooks 1, locking tab 2, locking clip 3, locking tab insertion component 4, and hinge 5. FIG. 3 shows side view of an exemplary device. Shown are wire hooks 1, locking tab 2, and living hinge 5. FIG. 4 shows a close up of the locking area of an exemplary device. Shown are locking tab 2, locking clip 3, and locking tab insertion component 4. FIG. 5 shows a close up of the locking area of an exemplary device. Shown are locking tab 2, rubber washer 6, and closing tab 7. FIG. 6 shows a close up of the inner track for wrapping and securing wires of an exemplary device. Shown are wire hooks 1, locking tab 2, locking clip 3, and hinge 5.

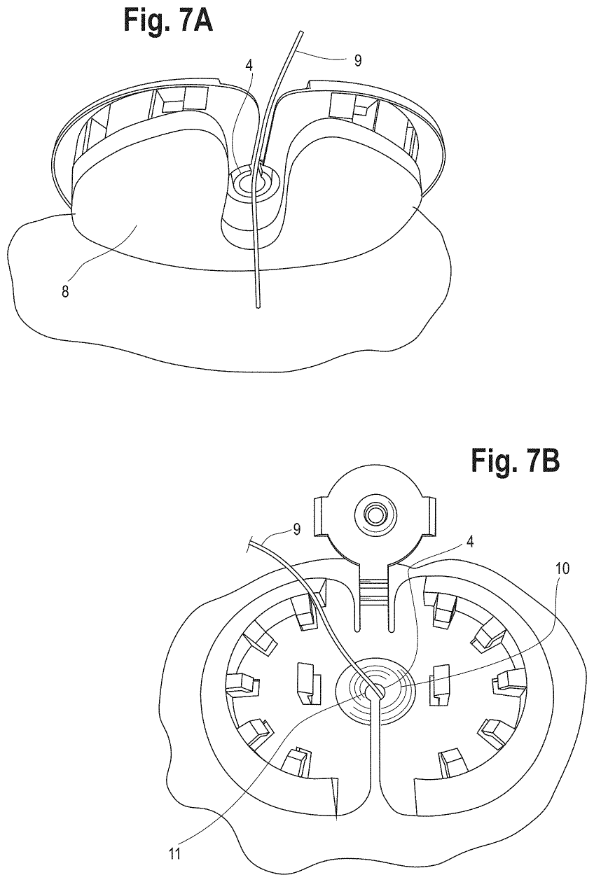

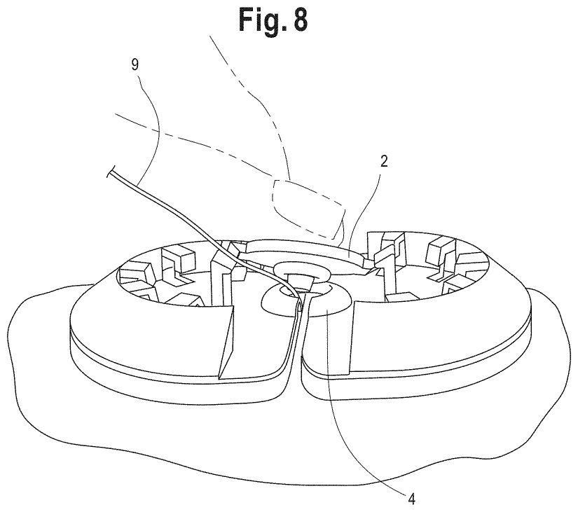

FIGS. 7-11 show exemplary devices in operation. FIG. 7 shows locking tab insertion component 4, optional adhesive and padding 8, and guide wires 9. The locking tab insertion component 4 further comprises a wire insertion groove 11 (e.g., a Y-shaped insertion groove 11) for inserting wire into hole 10. The padding 8 is constructed from any suitable material (e.g., foam). In some embodiments, the padding 8 comprises adhesive to attach the device to the patient's body. The guide wires 9 are inserted through the hole in locking tab insertion component 4. After the guide wire 9 is inserted through the hole 10 in locking tab insertion component 4, the locking tab 2, is closed by inserting into locking tab insertion component 4 as shown in FIG. 8.

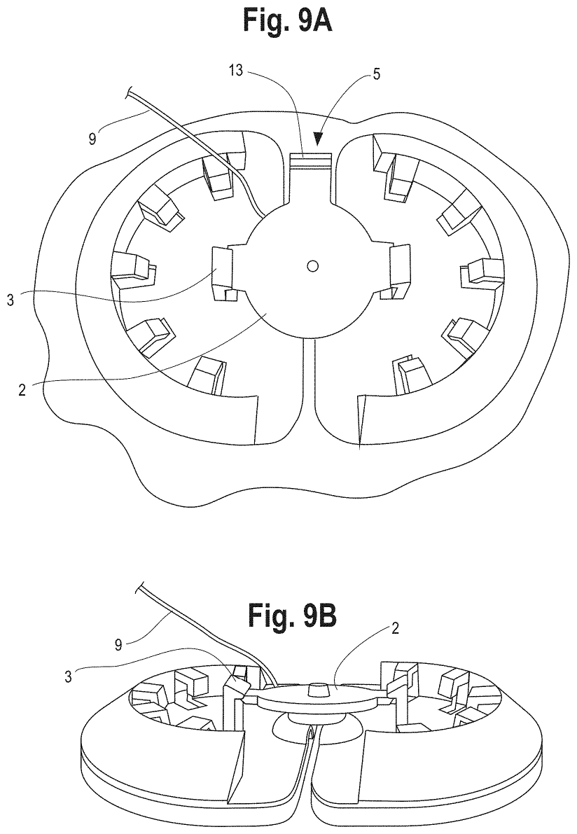

FIG. 9 shows guide wires 9 secured in an exemplary device. The locking tab 2 is closed and secured with locking clip 3, sealing the guide wires in the hole 10 in the locking tab insertion component 4. The left panel of FIG. 9 shows a top view of the closed device with guide wire 9 exiting the back lower section of the device, although other exit points are specifically contemplated. The right panel shows a side view of the closed device with the guide wire 9 exiting the back lower section of the device.

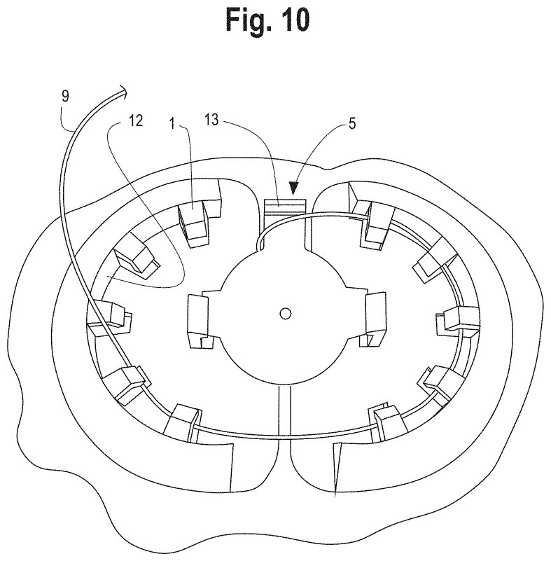

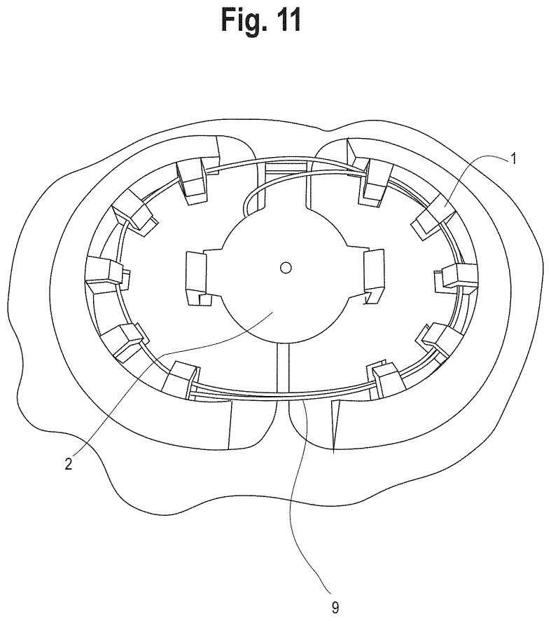

FIG. 10 shows how guide wires 9 are secured in the closed device. After securing the guide wires 9 in the device, the guide wire 9 are wrapped in the grooved track 12 about wire hooks 1 to further secure the guide wires 9. FIG. 11 shows guide wires 9 fully secured on wire hooks 1. The patient is now free to move without the guide wires 9 dislodging or shifting. Prior to surgery (e.g., in the pre-op or operating room), the guide wires 9 are removed from the device by removing guide wires 9 from the wire hooks 1, opening locking tab 2, and removing guide wires 9 from the hole in locking tab insertion component 4.

Devices described herein are constructed of any suitable material (e.g., polymers (e.g., polypropylene or polyethylene polymers), plastics, resins, etc.). In some embodiments, devices are fabricated from a mold or cast. In some embodiments, devices are injection molded or reaction injection molded, although other methods are specifically contemplated (e.g., vacuum (thermal) forming or compression molding).

The methods and devices described herein find use in securing guide wires for any number of non-limiting uses. In some embodiments, the guide wires are surgical guide wires (e.g., to identify a lump, lymph node (e.g., axilla, inguinal, or neck lymph nodes), soft tissue nodule, or suspected tumor). In some embodiments, the guide wires are used by surgeons to find non-palpable lumps for biopsy and/or lumpectomy (e.g., breast cancer or other tumors) located in any body part or tissue (e.g., chest wall, breast, abdomen, back, adipose tissue, muscle, lymph node, breast, etc.). Additional uses are specifically contemplated.

In some embodiments, wire management devices described herein further comprise a medical device communication system. In particular, provided herein is a communication device (e.g., for use with and as part of a wire management device) that provides secure wireless data storage and transmission of medical device and specific in vivo image information.

Presently there are no solutions that quickly allow providers to access device specific ID needed to ensure quick or safe use outside their local areas. The present disclosure minimizes these deficiencies, allows quick and accurate device identification, specific medical images, device use parameters, and patient diagnosis specifics, which alleviates uncertainty, improves patient safety, and enables device utility.

The present disclosure provides a wire management device comprising a computer readable medium chip that triggers and/or enables a patient approved provider's electronic device to access a precision IR of a compendium of device and patient relevant, specific medical images related to the device, specific device ID, device age, class and use/limitation parameters. In some embodiments, the specific patient diagnoses and allergies are also stored. In some embodiments, information is read only and complies with all HIPPA and DICOM standards.

In some embodiments, the described devices and methods do not replace original PACS and EMR records, but provide quick, specific, and powerful add-on systems as patients travel outside the location of original device placement.

Accordingly, provided herein is a composition, comprising: an wire management device as described herein comprising a computer readable medium comprising a) a wireless communications protocol and one or more of a) information about the device; b) images of the device; and c) a device specific identification number that pairs with a remote server.

The present disclosure is not limited to particular information. Examples include, but not limited to, device ID, device age, device brand, device class and use parameters, patient information (e.g., one or more of facility (e.g., original facility) information, patient name, age, and date of birth, diagnosis information, patient allergies, treatment information, or other medically relevant health information), or device model. In some embodiments, the images comprise images of the device in place in a subject.

The present disclosure is not limited to a particular image type or format. In some embodiments, images are mammograms, ultrasounds, X-rays, CT scans, MRIs, or other imaging modalities. In some embodiments, the images are digital Imaging and Communications in Medicine (DICOM) images. DICOM is the standard for the communication and management of medical imaging information and related data. DICOM is most commonly used for storing and transmitting medical images enabling the integration of medical imaging devices such as scanners, servers, workstations, printers, network hardware, and picture archiving and communication systems (PACS) from multiple manufacturers. It has been widely adopted by hospitals and is making inroads into smaller applications like dentists' and doctors' offices.

DICOM files can be exchanged between two entities that are capable of receiving image and patient data in DICOM format. In some embodiments, the different devices come with DICOM Conformance Statements, which state which DICOM classes they support. The standard includes a file format definition and a network communications protocol that uses TCP/IP to communicate between systems.

In some embodiments, the information informs a user of a compendium of specific device and patient relevant information, data, and images. Such a compendium provides a user (e.g., provider) with a concise and precise curated set of relevant information that saves time and improves care.

In some embodiments, the computer readable medium comprises a communication component selected from, for example, a radiofrequency identification chip, a near field communication chip, a blue tooth communication chip, or a WiFi communication chip. Radio-frequency identification (RFID) uses electromagnetic fields to automatically identify and track tags attached to objects. An RFID tag consists of a tiny radio transponder; a radio receiver and transmitter. When triggered by an electromagnetic interrogation pulse from a nearby RFID reader device, the tag transmits digital data, usually an identifying inventory number, back to the reader. This number can be used to inventory goods. There are two types. Passive tags are powered by energy from the RFID reader's interrogating radio waves. Active tags are powered by a battery and thus can be read at a greater range from the RFID reader; up to hundreds of meters. Unlike a barcode, the tag doesn't need to be within the line of sight of the reader, so it may be embedded in the tracked object. RFID chips are commercially available from a variety of suppliers (e.g.,

Near-field communication (NFC) is a set of communication protocols by which two electronic devices communicate when they are brought within 4 cm (112 in) of each other. NFC devices are used in contactless payment systems, similar to those used in credit cards and electronic ticket smart cards, and allow mobile payment to replace or supplement these systems. This is sometimes called NFC/CTLS and CTLS NFC, with contactless abbreviated CTLS. NFC is used for social networking and for sharing contacts, photos, videos, and other files. NFC devices can act as electronic identity documents and keycards. NFC offers a low-speed connection with simple setup that can be used to bootstrap more-capable wireless connections. The Secure Element chip is an NFC chip that contains data such as the Secure Element Identifier (SEID) for secure transactions. This chip is commonly found in smartphones and other NFC devices.

Bluetooth is a wireless technology standard used for exchanging data between fixed and mobile devices over short distances using short-wavelength UHF radio waves in the industrial, scientific and medical radio bands, from 2.400 to 2.485 GHz, and building personal area networks (PANs). It was originally conceived as a wireless alternative to RS-232 data cables.

Wi-Fi is a family of wireless networking technologies, based on the IEEE 802.11 family of standards, which are commonly used for local area networking of devices and Internet access. Wi-Fi uses multiple parts of the IEEE 802 protocol family, and is designed to interwork seamlessly with its wired sibling Ethernet. Compatible devices can network through a wireless access point to each other as well as to wired devices and the Internet. The different versions of Wi-Fi are specified by various IEEE 802.11 protocol standards, with the different radio technologies determining radio bands, and the maximum ranges, and speeds that may be achieved. Wi-Fi most commonly uses the 2.4 gigahertz (120 mm) UHF and 5 gigahertz (60 mm) SHF ISM radio bands; these bands are subdivided into multiple channels. Channels can be shared between networks, but only one transmitter can locally transmit on a channel at any moment in time.

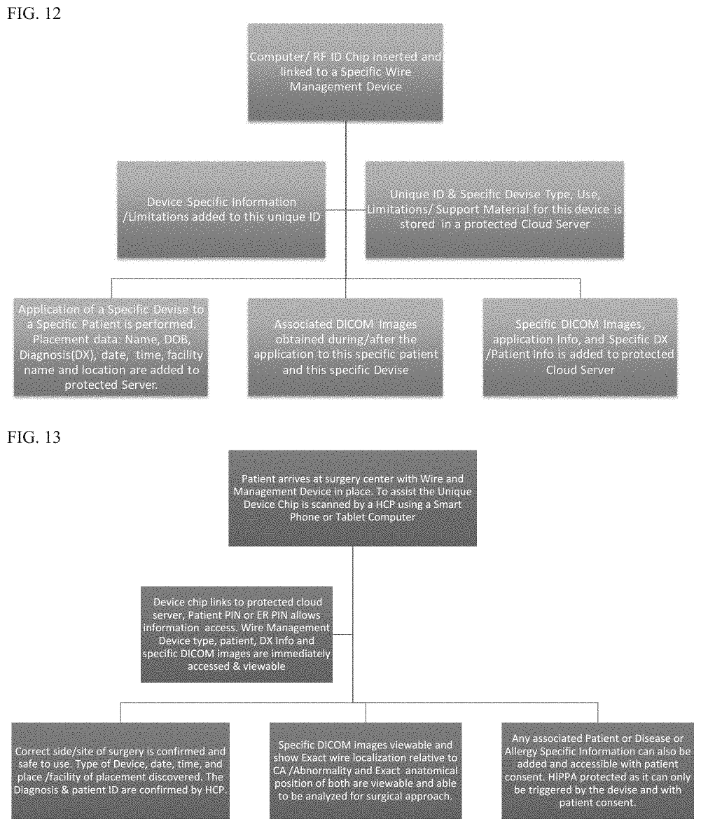

FIGS. 12-13 show exemplary workflows for device, systems, and methods of embodiments of the disclosure. FIG. 12 shows device centric and device triggered patient specific storage of DICOM Images, clinical application information, and device use/limitation specifics. A RF ID or other chip with a unique ID is inserted and linked to the device. Next, device specific information is added to this unique ID. The unique ID and device information is stored in a protected server or on the chip. In some embodiments, these steps are conducted prior to placement of the device in or on the subject. The device is then assigned and placed in or on the subject. Information on the subject (e.g., name, date of birth, time of placement, facility name and location, etc.) are added to the device information on the server or chip. In some embodiments, associated images (e.g., DICOM images) obtained during or after application of the device to the subject are also added to the device information on the chip or server.

FIG. 13 shows device centric and device triggered patient specific retrieval of DICOM Images, clinical application information, and device use/limitation specifics. A subject arrives at a surgery center with the wire management device in place. The provider scans the device (e.g., using a smart phone, tablet, or other portable device) to assess the unique device ID and associated information. The device chip provides or links to the remote server to obtain information and images. In some embodiments, a patient specific or provider specific PIN or passcode is required to access information. The provider is then able to confirm device information and correct placement/usage of device as well as correct side/site/conformation and best approach to surgical excision.

In some embodiments, the information is stored on the computer readable medium or on a remote server. In some embodiments, the computer readable medium is integrated into an external or internal portion of the device. In some embodiments, the information is stored in a read-only format.

In some embodiments, the secure storage of information is on the computer readable medium or in a privacy protected computer server that is accessible wirelessly. Examples include, but are not limited to, a third-party protected cloud site, or a device manufacturer protected site. In some embodiments, as described herein, information is accessible only by patient consent, only by scanning of the patient applied device with the ID chip, and/or only with proper approved passcodes.

As described herein, in some embodiments, information stored on the medical device is accessed by a personal electronic device. The present disclosure is not limited to particular personal devices. Examples include, but are not limited to, a smart phone, smart watch, tablet, or laptop computer.

To trigger, sort, and retrieve the needed information at a later date from other places around the world, the process is quick and quite simple. The patient allows a health care provider to a bring a tablet computer, smartphone, RF-ID reader, laptop computer or other personal device within range (e.g., 2 inches to multiple feet depending on the communication technology of the device) so it can read the chip information by NFC, RF-ID, Blue tooth, or WiFi. The patient may also need to supply a passcode (e.g., PIN) during the process. The wireless device then allows the medical device to trigger, sort, and retrieve the above information, allowing the personal device display the specific device documents, application DICOM images and any included medical diagnoses, or added patient information. In some embodiments, the access by wireless devices is read-only.

In some embodiments, access to information requires a patient specific passcode to the personal electronic device. In some embodiments, the passcode is required for access to the information. In some embodiments, patients pre-approve and sign for device triggered retrieval of specific diagnostic images, device specifics, and diagnoses information to outside providers in urgent/emergent times, or in cases where the patient is in need (e.g., outside their usual region of intervention/treatment).

Once viewed by a medical health care provider, surgeon, or radiologist, information is read, but not copied or forwarded, and not printed. In some embodiments, it is possible to add to the stored file images or medical diagnoses, and interventions if the patient agrees. However, in some embodiments, nothing can be edited out or altered from the past device centric record. For example, in some embodiments, previous information, along with new ongoing diagnoses, data, images, can be added, triggered, accessed, and read by future caregivers at any distant sites as needs arise.

In some embodiments, the data storage, access, and communication compositions and methods described herein are in compliance with the Health Insurance Portability and Accountability Act of 1996 (HIPAA or the Kennedy-Kassebaum Act). The HIPAA Privacy Rule is composed of national regulations for the use and disclosure of Protected Health Information (PHI) in healthcare treatment, payment and operations by covered entities. The HIPAA Privacy Rule regulates the use and disclosure of protected health information (PHI) held by “covered entities” (generally, health care clearinghouses, employer-sponsored health plans, health insurers, and medical service providers that engage in certain transactions). By regulation, the HHS extended the HIPAA privacy rule to independent contractors of covered entities who fit within the definition of “business associates”. PHI is any information that is held by a covered entity regarding health status, provision of health care, or health care payment that can be linked to any individual. This is interpreted rather broadly and includes any part of an individual's medical record or payment history. Covered entities must disclose PHI to the individual within 30 days upon request. Also, they must disclose PHI when required to do so by law such as reporting suspected child abuse to state child welfare agencies.

A covered entity may disclose PHI to certain parties to facilitate treatment, payment, or health care operations without a patient's express written authorization. Any other disclosures of PHI require the covered entity to obtain written authorization from the individual for the disclosure. In any case, when a covered entity discloses any PHI, it must make a reasonable effort to disclose only the minimum necessary information required to achieve its purpose. Under HIPAA, HIPAA-covered health plans are now required to use standardized HIPAA electronic transactions. See, 42 USC § 1320d-2 and 45 CFR Part 162. Information about this can be found in the final rule for HIPAA electronic transaction standards (74 Fed. Reg. 3296, published in the Federal Register on Jan. 16, 2009), and on the CMS website.

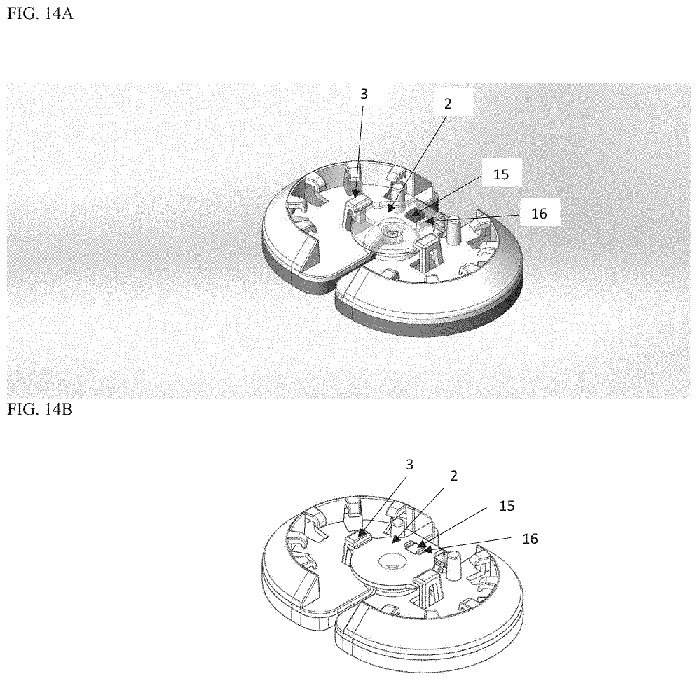

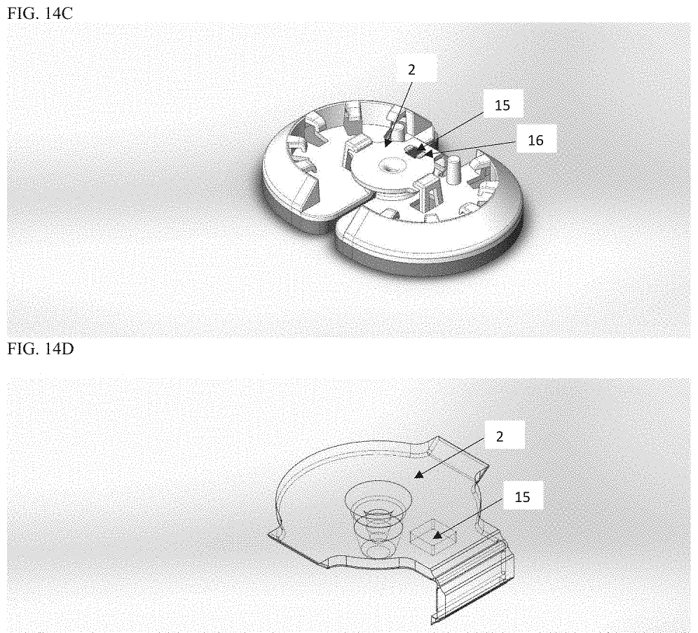

FIGS. 14A-D shows exemplary guide wire management devices comprising a computer readable medium chip (CRM chip). FIG. 14A shows a locking tab 2 closed and secured with locking clip 3. The locking tab 2 is transparent to shown CRM chip 15 and CRM chip securing tabs 16. In the embodiment shown in FIG. 14A, the CRM chip 15 is attached to the base of the device underneath locking tab 2.

FIG. 14B shows an embodiment of a guide wire management device where a CRM chip is embedded in locking tab 2 via CRM chip securing tabs 16. In FIG. 14B, the locking tab 2 is closed and secured with locking clip 3. The CRM securing tabs 16 keep the CRM chip 15 in place regardless of the position of locking tab 2. In FIG. 14B, the CRM chip 15 is not engaged in CRM securing tabs 16.

FIG. 14C shows the guide wire management device of FIG. 14C with CRM chip 15 engaged with CRM securing tabs 16. In the embodiment shown in FIG. 14C, locking tab 2 is closed and secured with locking clip 3. The CRM securing tabs 16 keep the CRM chip 15 in place regardless of the position of locking tab 2.

FIG. 14D shows an embodiment of a guide wire management device where a CRM chip 15 is embedded in locking tab 2 without any additional securing component. For example, in some embodiments, the CRM chip 15 is added during manufacture (e.g., molding) or a slot or hole is machined in locking tab 2 after manufacture.

The present disclosure is not limited to the physical locations of computer readable medium chips on devices shown in the figures. Any suitable location on the medical device is specifically contemplated.

In some embodiments, the information is used to recommend, determine, or administer a medical course of action (e.g., whether or not to proceed with surgery and the proper location of surgery). In some embodiments, the treatment course of action is administered to the patient or subject.

All publications and patents mentioned in the above specification are herein incorporated by reference as if expressly set forth herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the following claims.

We claim:

1. A guide wire securing device, comprising:
a computer readable medium comprising a) a wireless communications protocol and one or more of a) information about the device; b) images of the device; and c) a device specific identification number that pairs with a remote server.

2. The guide wire securing device of claim 1, wherein said guide wire securing device comprises i) a wire retention and management component comprising a plurality of wire hooks and a grooved wire track formed by said wire hooks that passes through all of said wire hooks, ii) a locking component positioned at a central portion of said device configured to secure a guide wire passed through said wire track to said central portion of said device, wherein said locking component comprises a locking tab, a locking tab insertion component configured to receive said locking tab, a living hinge configured to allow said locking tab to bend, a wire insertion groove comprising a wire hole and configured to receive said guide wire from said wire retention and management component, and a locking clip configured to secure said locking tab in the closed position, and iii) an attachment component configured to secure said device to a subject, wherein said wire hooks are arranged in a circle or semi-circle around said locking component.

3. The guide wire securing device of claim 2, wherein said wire insertion groove is Y-shaped.

4. The guide wire securing device of claim 2, wherein said locking tab comprises a rubber washer and closing tab.

5. The guide wire securing device of claim 2, wherein said attachment component comprises adhesive attached to a back edge of said device.

6. The guide wire securing device of claim 5, wherein said adhesive is foam adhesive.

7. The guide wire securing device of claim 2, wherein said device is fabricated from a polymeric material.

8. The guide wire securing device of claim 1, wherein said information is selected from the group consisting of device ID, device age, device brand, device class and use parameters, and device model.

9. The guide wire securing device of claim 1, wherein said images comprise images of the device in place in a subject and/or other relevant patient medical images.

10. The guide wire securing device of claim 1, wherein said computer readable medium comprises a communication component selected from the group consisting of a radiofrequency identification chip, a near field communication chip, a blue tooth communication chip, and a WiFi communication chip.

11. The guide wire securing device of claim 1, wherein said information is stored on said computer readable medium or on said remote server.

12. The guide wire securing device of claim 1, wherein said computer readable medium is integrated into an external or internal portion of said device.

13. The guide wire securing device of claim 1, wherein said computer readable medium further comprises patient information.

14. The guide wire securing device of claim 13, wherein said patient information comprises one or more of name, date of birth, facility information, diagnosis information, treatment information, allergy information, and health information.

15. The guide wire securing device of claim 1, wherein said information is stored in a read-only format.

16. The guide wire securing device of claim 1, wherein said computer readable medium is affixed to the interior or the exterior of said device.

17. A system, comprising:
a) the guide wire securing device of claim 1; and
b) a personal electronic device configured to access information stored on said device.

18. The system of claim 17, wherein said personal device is a smart phone, smart watch, tablet, or laptop computer.

19. A method of retrieving medical device information, comprising:
a) contacting the guide wire securing device of claim 1 with a personal electronic device; and
b) accessing information stored on said medical device with said personal electronic device using said wireless communication protocol.

20. The method of claim 19, wherein said information informs a user of a compendium of specific device and patient relevant information, data, and images.
